(19) United States
(12) Patent Application Publication
(10) Pub. No.: US 2026/0198655 A1
(43) Pub. Date: Jul. 16, 2026

(54) ADAPTIVE FOREFOOT SHOE SUPPORT LAST

(71) Applicant: SHAPE REGIME APS, Tilst (DK)

(72) Inventor: Roald Nore, Risskov (DK)

(21) Appl. No.: 19/138,239

(22) PCT Filed: Dec. 15, 2023
(86) PCT No.: PCT/DK2023/050314

§ 371 Date: Jun. 12, 2025

(30) Foreign Application Priority Data
Dec. 15, 2022 (DK) PA202270620

(51) Int. Cl. A43D 3/14; A61L 9/12

(52) U.S. Cl. CPC A43D 3/14; A61L 9/12

(57) ABSTRACT

A support shoe last for supporting a shoe is provided, the support shoe last includes —a shell body defining a forepart, a waist part including a cone part, and a feather edge between the forepart and the waist part, the shell body includes —a top section extending from the waist part to forepart; —an arch side section and an outer side section extending downwards from the top section, thereby defining a cavity of the shell body; and —an arch landing pad configured to engage an insole of a shoe, where the arch landing pad extends from part of the arch side section and widens towards the insole, —a second landing pad configured to engage an insole of a shoe, the second landing pad extends from part of the outer side section and widens towards the insole, wherein the arch side section and/or the outer side section comprise a mechanical weakness.

### CROSS REFERENCE TO RELATED APPLICATIONS

This application is a national stage of PCT Application No. PCT/DK2023/050314, having a filing date of Dec. 15, 2023, which is based on DK Application No. PA 2022 70620, having a filing date of Dec. 15, 2022, the entire contents both of which are hereby incorporated by reference.

### FIELD OF TECHNOLOGY

The following relates to support shoe last for supporting a shoe on display or during transportation.

### BACKGROUND

Shoes are manufactured in specific anatomical shapes, but the original shape of the shoe collapses over time. Thus, the shoe needs to be supported when it is not in use to prevent it from losing its original shape and prolong the time it holds its original shape. Furthermore, a shoe on display without a shoe support last is not representative of how a shoe looks when worn i.e. the tongue of the shoe is for example poorly presented. Support shoe lasts for supporting the shoe when not in use are therefore well known within the art.

Conventional art support shoe lasts are full foot support shoe lasts that comprise of a front and a back piece connected by an adjustable middle piece, where the front piece fills the forepart of the shoe and the back piece pushes against the heel grip of the shoe to hold the front piece in place. The middle piece is often a spring. These conventional art support shoe lasts are made of inflexible materials such as wood or rigid plastic materials, which result in support shoe lasts that are rigid and only flexible in the length of the last because of the adjustable middle piece.

The problem with conventional art support shoe lasts is that they have an incorrect shape, in that it is not anatomically correct, thereby being unable to support the original anatomical shape of the shoe. Another problem originates from the rigid front piece being pushed into the forepart of the shoe by the spring and back piece which apply an unnatural upwards pressure on the tongue of the shoe, which along with the incorrect shape deforms the shoe over time instead of supporting the original shape of the shoe. The insertion of the support last into the shoe often requires an excessive force, which further deforms the shoe by applying an incorrect pressure on the shoe.

Additionally, when the shoes are displayed at retails, in showrooms, at fairs, or by re-sellers, the shoe should appear true to original shape and weight. This is not possible with conventional art support shoe lasts, as they are inflexible and thus not adaptive to the original shape of the shoe, and they are often also heavy due to the materials they are made of.

When the shoes are delivered from the manufacturer they are filled with paper or cardboard to support the shape of the shoe. The problem with using disposables for shoe support is that it is not sustainable. For example, in China more than 12 billion pairs of shoes are produced a year, which result in enormous amounts of disposable filling.

There is a need in the art for a support shoe last that is flexible and adaptive to the anatomic shape of the shoe, thereby imitating the original shape of the shoe and preventing the shoe from losing its original shape. There is also a need for a support shoe last that is easy to insert and light weighted. Finally, there is a need for a support shoe last that can replace the disposable paper and cardboard fillings, used by the manufacturer, for a more sustainable support solution.

### SUMMARY

An aspect relates to a support shoe last capable of adjusting to different shapes of different shoes so that the support shoe last does not deform the shoe similar to conventional art support shoe lasts.

An aspect of embodiments of the invention is achieved by a support shoe last for supporting a shoe on display or during transportation. The support shoe last comprises a shell body defining a forepart, a waist part comprising a cone part, and a feather edge between the forepart and the waist part. The shell body comprises
a top section extending from the waist part to the forepart;
an arch side section and an outer side section extending downwards from the top section, thereby defining a cavity of the shell body; and
an arch landing pad configured to engage an insole of a shoe, the arch landing pad, where the arch landing pad extends from part of the arch side section and widens towards the insole,
a second landing pad configured to engage an insole of a shoe, the second landing pad extends from part of the outer side section and widens towards the insole,
   wherein the arch side section and/or the outer side section comprises a mechanical weakness extending longitudinal along at least part of the shell body, thereby enabling the arch section and the outer side section to bend and adjust to the shape of a shoe.

The support shoe last may support a shoe comprising an outsole in contact with a surface on which the shoe is placed, and an upper part of the shoe that covers the foot that extends upwards from the outsole. The upper part may comprise a toe cap and a vamp, where the vamp is in connection with a tongue at the front of the shoe, and quarters at the rear of the shoe. The quarters may cover the heel of the foot. An insole in parallel contact with the outsole may comprise the bottom of the inside of the shoe. The shoe will further comprise a shoe opening where the foot can be inserted into the shoe.

The support shoe last may be inserted into the shoe opening and placed in the shoe such that the shell body supports the toe cap, vamp, and tongue of the shoe. Thus, the support shoe last may support the front of shoe from the inside of the shoe. Thereby, the support shoe last is a front foot support shoe last.

The shell body of the support shoe last that defines a forepart, a waist part and a feather edge may be constructed to simulate the anatomy of a front foot. The shell body enables the support shoe last to simultaneously support the shoe while the support shoe last is reshaped by the shoe such that the support shoe last does not permute the shoe in an unnatural way as would the conventional art support shoe lasts which are made of solid plastic or wood which causes unwanted displacement of parts of the shoe unless the support shoe last is specifically designed to a particular shoe. A support shoe last is never designed to a specific shoe as this would be too expensive and it would be a waste of material, however the support shoe last according to embodiments of the present invention can support various different shoes by reshaping the support shoe last due to the mechanical weaknesses enabling the distance between the two landing pads to decrease as a function of the inner shoe shape.

The top section extending from the waist part to the forepart of the shell body will be in contact with the upper part of the front of the shoe. Thus, the arch side section and the outer side section extend downwards from the top section towards the insole of the shoe. Thereby, the shell body of the support shoe last is adapted to a curvature of the upper part of the front of the shoe.

The cavity of the shell body formed by the arch side section and the outer side section is hollow and enables the shell body to adapt to the shape of the shoe, i.e. the shell body makes the support shoe last flexible, and thus adaptive to the original shape of the shoe. A further advantage of this is that less material is needed to produce the support shoe last, thereby decreasing the price of the shoe last.

Another advantage is that the support shoe last becomes light weighted. Thereby, the support shoe last adds minimal weight to the shoe, which is an advantage in e.g., retail stores where the displayed shoes should appear true to the actual weight of the shoe, and during e.g., transportation of the shoe where a minimal weight contribution from the support shoe last is of interest, both with regard to manufacturers and travelling consumers.

An arch landing pad extends from the arch side section towards the outer side section along part of the waist part of the shell body and a second landing pad extends from the outer side section towards the arch side section along part of the waist part of the shell body, wherein both the arch landing pad and the second landing pad substantially lie in the same horizontal plane and both landing pads have an insole facing side which are in contact with the insole of the shoe.

The arch landing pad and the second landing pad may be asymmetrical in shape and in length. Thereby, the support shoe last can simulate the anatomic shape of a foot which is asymmetrical, and thus simulate the shape of a shoe which is shaped after a foot, and therefore also asymmetric and anatomic. The arch landing pad in connection with the arch side section may have a smaller radius of curvature towards the second landing pad, than the outer side section and the second landing pad; this configuration has in test shown to be optimal for support shoe lasts' adaptability.

The arch landing pad may extend from the waist to the feather waist of the shell body. On the contrary, the outer side section may be substantially straight deflecting in an outwards angle from the arch side section with an inwards curvature in connection with the second landing pad towards the arch landing pad. The second landing pad may extend from the waist to the forepart of the shell body. In some embodiments, the second landing pad is longer than the arch landing pad.

An advantage of this is that the support shoe last can better imitate the original anatomic shape of the shoe.

The arch side section and the outer side section each comprise a mechanical weakness extending longitudinal along at least part of the shell body, where the mechanical weakness may be achieved by decreasing the material thickness of the shell body on each side of the top section at the joint between the top section and each of the two side sections. Thereby increasing the flexibility of the support shoe last as various shoes will have different widths.

The support shoe last can be used for various shoe sizes as the dimensioning of the shoe support last just needs to be adapted to the different shoe sizes.

The mechanical weakness enables the arch section and the outer side section to bend inwards towards each other, thereby moving the arch landing pad and the second landing pad towards each other. The mechanical weakness further enables the support shoe last to adjust to the shape of a shoe. Thus, when inserting the support shoe last into the shoe, the arch landing pad and the second landing pad will automatically slide together and adjust to the width and height of the shoe.

An advantage of this is that the support shoe last is flexible and can better adapt to the original shape or natural shape of the shoe. Thereby providing the shoe with a natural support that does not deform the shoe or at least deform the shoe less than the conventional art, compared to conventional art support shoe lasts that are unable to adapt to the original shape of the shoe and apply and unnatural pressure to the shoe consequently deforming the shoe over time. Furthermore, it minimizes the collapsing of the shoe that appears over time.

In some embodiments the mechanical weakness of each side section may extend longitudinal along the entire shell body. However, this will also decrease the biasing force of the support shoe last, and this will increase the risk of the hollow body collapsing, thus these embodiments may be less desirable.

In some embodiments the mechanical weakness may extend longitudinal along a part of the shell body between the feather edge to the waist part. Thereby increasing the stability of the support shoe last and preventing collapse of the support shoe last under pressure, and still providing the necessary flexibility to the support shoe last.

The flexible construction of the support shoe last enables it to adapt to different types of shoes and to different shoe sizes within a specific range. When a shoe is worn it moulds to the shape of the user's foot over time. Thus, the support shoe last can furthermore adapt to the shape of a shoe changing over time and ensure that it maintains the shape of the user's foot. As the skilled person would know, it will not be possible to design a support shoe last for a size 36 and 47, but the support shoe last of embodiments of the present invention can be designed such that one size of the support shoe last can be used for at least 35-37 or 38-40 or 41-43 or 44-46 and so on.

The support shoe last may be made in a long-lasting and light material and may be a one-piece construction without any hollow or delicate parts, which make the support shoe last washable. Thus, the support shoe last is suitable for long time use and recyclable use.

An advantage of this is that the support shoe last is a more sustainable shoe support and filling, that can replace the disposable paper and cardboard fillings used by the manufacturers. The support shoe last is just as easy to insert into the shoe as paper and cardboard filling, thus, the production time is not prolonged by changing to the more sustainable support shoe last. Furthermore, the light weighted support shoe last will not increase the transportation costs.

The support shoe last may further be made in recyclable materials.

The support shoe last may be a forefoot support shoe last, thereby only filling and supporting the toe cap, vamp, and tongue of the shoe. Thus, the support shoe last is not visible in the shoe when inserted in the shoe. The support shoe last of embodiments of the present invention does not use spring elements extending from the forefoot to the backpart of the shoe for creating a biasing force to secure the support shoe last to the forefront. The spring element is often used for wood shoe lasts because a wood shoe last has a very smooth surface causing the shoe last to slide if not for the spring element. The spring element will deform the shoe in an unnatural way i.e. in a way which cannot happen when placing a foot inside the shoe.

An advantage of the support shoe last, is that when the shoe is on display in e.g., retail, showrooms, fairs or by re-sellers, then the support shoe last is not visible.

The visibility is also an advantage for the commercial photographing of shoes, as filling of the shoe is essential for the appearance of the shoe. Thus, the support shoe last being invisible from the outside when inserted into the shoe would significantly reduce the work of retouching the photographs of the shoe.

Another advantage of the support shoe last further is that it makes shoes on display in e.g., retail, showrooms, fairs or by re-sellers appear in their intended original shape and weight, due to its flexibility and light weight.

In an aspect of the support shoe last, wherein the landing pads may extend along part of the waist part to the feather edge and optionally along part of the forepart. The feather edge is the transition between the waist part and the forepart in most embodiments the forepart should be designed to adapt to the forepart and thus the forepart should not as such provide structural strength to the support shoe last that is the purpose of the waist part. However, the forepart may optionally have landing pads extending along at least part of the forepart from the feather edge. In some embodiments only the second landing pad will extend along part of the forepart, thereby forming an asymmetry between the two landing pads.

The landing pads will extend from the feather edge or substantially from the feather edge towards a rear side section of the hollow body which rear side section will be distal to the forepart. How long the landing pads must extend depends on the size of the shoe support last and how stiff one wants the shoe support last to be, however, this can be decided by trial and error by the skilled person.

Thereby supporting the upper part of shoe along the sides.

In an aspect of the support shoe last, the landing pads may comprise curved outer surfaces, which curved outer surfaces are curved inwardly towards the cavity. Thereby, the landing pads, the arch side section and outer side section will automatically deform inwardly towards the cavity of the shell body.

An advantage of this is that when the support shoe last is inserted in the shoe, the landing pads automatically squeezes together, due to the flexible construction and the inwards curvature, to adapt to the volume and fit of the shoe. Thereby adapting to and imitating the shape of the shoe.

In some embodiments, the landing pads may be curved upwards. The upwardly curved landing pads may slide together when the support shoe last is inserted into a shoe, thereby adjusting the support shoe last to the width and height of the shoe.

The upward curvatures may enable a smooth insertion of the support shoe last into the shoe, where scratching of the insole of the shoe and the support shoe last getting stuck is avoided. Thus, the upwardly curvature decreases the risk of the landing pads gripping the insole or other parts of the shoe.

An advantage of the landing pads curving upwardly is that the curvature ensures that the two landing pads do not collide with each other when pushed together inside the shoe.

Here, upwardly is seen relative to an insole of a shoe standing on a surface when the shoe support last is used as intended.

In an aspect of the support shoe last, the curvature of the outer surface of the second landing pad may be greater than the curvature of the outer surface of the arch landing pad. This will cause the arch landing pad to deform to the shape of the shoe before the second landing pad as this results in a more natural fit wherein the shoe is not deformed in a way which will not happen when inserting a foot.

In an aspect of the support shoe last, wherein the shell body may further comprise at the waist part and distal to the forepart, a rear side section extending downwardly from the top section and interconnecting the arch side section and the outer side section. This will provide a stiffness to the shell body and increase the stability wherein collapse of the support shoe last under pressure is prevented.

The rear side may further be constructed without a mechanical weakness, which provides additional stiffness to the shell body. Thereby further increasing the stability of the support shoe last and preventing collapse of the support shoe last under pressure.

An advantage of the rear side section is that the support shoe last can support shoes packed in e.g., bags or suitcases and resist the pressure from the other content in the bags or suitcases.

The rear side section may further be constructed so that a gap is obtained between the rear side and the insole of the shoe, thereby enabling the rear side to act as a handle, that two fingers may grab around for easy removal of the support shoe last from the shoe.

In some embodiments the top of the rear side may be constructed with a tip peaking at a lower point than the highest point of the top section. This tip may not be configured to interact with the shoe and thus not serve a support purpose in the shoe. Instead, it eases the removal of the support shoe last from the shoe as a finger may grab around the tip, thereby providing a better grip when pulling the support shoe last out of the shoe.

In an aspect of the support shoe last, wherein the mechanical weakness is formed by having less material thickness compared to the average material thickness of the side sections and/or formed by micro-perforations or macro-perforations along the side section.

The macro-perforations may have the dimensions 2×3 cm or 1.5×2.5 cm or 1×2 cm similar sizes in the range of cm.

The micro-perforations may have a diameter of 2-7 mm or 3-5 mm or 3 mm or 4 mm or 5 mm.

Forming the mechanical weakness by decreasing the material thickness of the of the side sections and/or making perforations along the side sections increases the flexibility of the bend of the two side sections, and less material is needed to produce the support shoe last. Thereby increasing the flexibility and decreasing the price of the support shoe last.

Decreasing the material thickness for forming mechanical weaknesses and/or perforations further decreases the weight of the support shoe last. Thereby, the support shoe last adds minimal weight to the shoe, which is an advantage in e.g., retail stores where the displayed shoes should appear true to the actual weight of the shoe, and during e.g., transportation of the shoe where a minimal weight contribution from the support shoe last is of interest, both when it regards manufacturers and travelling consumers.

An advantage of the micro-perforations or macro-perforations is that they may increase diffusion of air from and into the shoe, thereby increasing the respiration of the shoe, prolonging the durability of the shoe, and preventing bad odor.

Another advantage of the micro-perforations or macro-perforations is that they provide texture to the surface of the support shoe last, thus securing the support shoe last within the shoe by increasing friction. Thereby, the support shoe last holds itself in place within the shoe. The friction from the micro-perforations is greater than the macro-perforations. Thereby, the support shoe last can be secured without the need for an additional spring.

In an aspect of the support shoe last, wherein the support shoe last may comprise thermoplastic material such as thermoplastic polyurethane or recycled thermoplastic polyurethane or plant-based thermoplastic polyurethane or polyethylene.

Thermoplastic polyurethane or recycled thermoplastic polyurethane or plant-based thermoplastic polyurethane may be used for making the support shoe last, as it is very flexible materials and can be made in different degrees of hardness. It is therefore possible to make varying thicknesses of the shell body, thereby creating mechanical weakness to increase the flexibility of the support shoe last without risking breakage.

An advantage of thermoplastic polyurethane or recycled thermoplastic polyurethane or plant-based thermoplastic polyurethane is that it is a washable, light weighted, and durable material.

An advantage of polyethylene is that it is a cheap material, and thus provides low production costs of the support shoe last, while providing sufficient strength and flexibility.

In some embodiments, the support shoe last may comprise of a single material and cast in a single mould.

The support shoe last may have a matt surface having a texture that provides the support shoe last with a light grip, thus securing the support shoe last within the shoe. Thereby, the support shoe last holds itself in place within the shoe.

In some embodiments, the support shoe last may comprise of two materials and cast in a dual mould.

In some embodiments the cone part of the shell body may have a higher surface roughness than the other part of at least the waist part and optionally higher surface roughness compared to the rest of the shell body, such that the support shoe last is secured within the shoe.

The cone part may be made of a softer thermoplastic polyurethane, and the rest of the shell body may be made of a harder but still flexible thermoplastic polyurethane. The softer thermoplastic polyurethane may have a higher surface roughness than the harder thermoplastic polyurethane. Thus, the cone part may provide friction between the support shoe last and the inner lining of the shoe. Thereby, the support shoe last holds itself in place within the shoe.

And advantage of this is that the support shoe last does not require a back piece pushing against the heel grip of the shoe as the conventional art support shoe lasts do. Thereby, an unnatural pressure on the shoe that deforms the shoe is avoided.

The harder thermoplastic polyurethane may have a smooth surface that slides smoothly along the inner lining of the shoe, which ensures an easy insertion and removal of the support shoe last.

The cone part may be connected to the rear side section, and in some embodiments the rear side section may comprise of the same material as the cone part. Thereby providing a better grip for pulling the support shoe last out of the shoe.

In an aspect of the support shoe last, wherein the forepart may comprise a toe spring. Thereby, the forepart will rise upwardly relative to a bottom surface of the feather edge and the waist part enables that the support shoe last can support many different shoe shapes as the toe spring will also result in less material and this enables the support shoe last to flex the forepart.

In an aspect of the support shoe last, wherein the forepart may comprise a split toe formed by a slit, wherein sides of the slit are curved downwardly.

Thereby, each side of the split will automatically move downwards and together and adjust to the shape of the toe of the shoe.

When the support shoe last is inserted in the shoe, the sides of the slit automatically squeeze together, due to the flexible construction and the downwards curvature, to adapt to the volume and fit of the shoe. Thereby adapting to and imitating the shape of the shoe.

An advantage of the sides of the slit curving downwardly is that the insertion of support shoe last into the shoe eases, because the curved sides slide smoothly along the inside of the shoe.

Another advantage of the sides of the slit curving downwardly is that the curvature ensures that the two sides do not collide with each other when pushed together inside the shoe.

The split toe may further increase the flexibility of the support shoe last, thereby enabling the support shoe last to narrow in the forepart.

An advantage of this is that the support shoe last can fit different types and sizes of shoes with both wide and narrow toe caps.

Here downwardly is seen relative to an insole of a shoe standing on a surface when the shoe support last is used as intended.

In an aspect of the support shoe last wherein the shell body may further comprise a fragrance holder configured to hold a fragrance plug-in. The fragrance plug-in reduces bad odor from the shoe. The fragrance plug-in may be replaceable such that the fragrance can be changed to another odor or be replaced when depleted.

An advantage of this is that it can prevent bad odor from spreading from shoes packed in e.g., a suitcase or a bag to the rest of the packed content.

In some embodiments the fragrance holder may be a tube with a cap for holding the fragrance plug-in, thereby reducing bad odor from the shoe.

In some embodiments the support shoe last has built-in clips inside the cavity on the backside of the top section, for holding the fragrance plug-in, thereby reducing bad odor from the shoe.

In an aspect, the fragrance holder may be positioned at the rear side section and optionally the fragrance holder may comprise a perforated tube extending through the rear side section, thereby making the fragrance holder accessible even when the support shoe last is supporting a shoe.

In some embodiments the fragrance holder may comprise a tube configured for hold a fragrance plug-in. The opening of the tube may be positioned at the rear side section and in some embodiments, it may be cast into the shell body of the support shoe last.

The tube may comprise a cap for closing of the opening of the tube and keeping the fragrance plug-in secured inside the tube.

The tube may further comprise tube perforations enabling diffusion of fragrance from the fragrance plug-in inside the tube to the shoe.

The support shoe last may comprise a tube aperture in the rear side section for receiving and holding the perforated tube such that the perforated tube can be changed or replaced.

In an aspect, the fragrance holder may extend through an aperture of the shell body, for example an aperture of the rear side section.

The main purpose of the fragrance holder is to be used together with the support shoe, however the fragrance holder can be separate from the support shoe last.

In an aspect, the fragrance holder may comprise
a tube with tube perforations, and
a cap comprising a cap cylinder extending from a cap flange, wherein the cap flange having a greater width than the cap cylinder,
   wherein the cap cylinder comprising a channel adapted for removable holding a fragrance stick, wherein the cap cylinder is configured to frictionally engage the tube.

The channel enables that only the fragrance stick needs to be replaced when the fragrance from the fragrance stick has evaporated, thereby waste is reduced. The channel also enables that the fragrance stick can be changed. The cap functions as a handle of the fragrance stick, such that fragrance stick can be handled without touching the fragrance stick.

In an aspect, the cap and/or the tube comprises a stop member such that there is distance between the cap flange and the tube when the cap is inserted partly into the tube. The purpose of the stop member is to prevent the cap or cap cylinder to be inserted completely into the tube as this will create a distance between the tube and the cap flange such that it is easier to remove the cap from the tube either using a finger or an object being wedge in between the cap and the tube.

In an aspect, the tube comprises a tube flange for engaging the shell body. Thereby, the tube cannot be pressed through the shell body. The same effect applies when engaging other holes.

In an aspect, the tube comprises one or more external friction members for frictionally engaging the aperture of the shell body. The one or more friction members may be one or more protrusions and/or material with a higher friction coefficient.

In an embodiment, the tube comprises two ring protrusions positioned with a predefined distance.

In an aspect, the fragrance holder may comprise a cord connector comprising a first connector aperture adapted for frictionally engaging the tube and a second connector aperture adapted for engaging a cord. Thereby, the fragrance holder can be used not only in connection with a support shoe last but also placed in a sports bag or a connected to a cord of a piece of cloth.

In an embodiment, the tube comprises two ring protrusions positioned with a predefined distance, which distance is substantially equal to the thickness of the cord connector.

### BRIEF DESCRIPTION

Some of the embodiments will be described in detail, with references to the following Figures, wherein like designations denote like members, wherein:

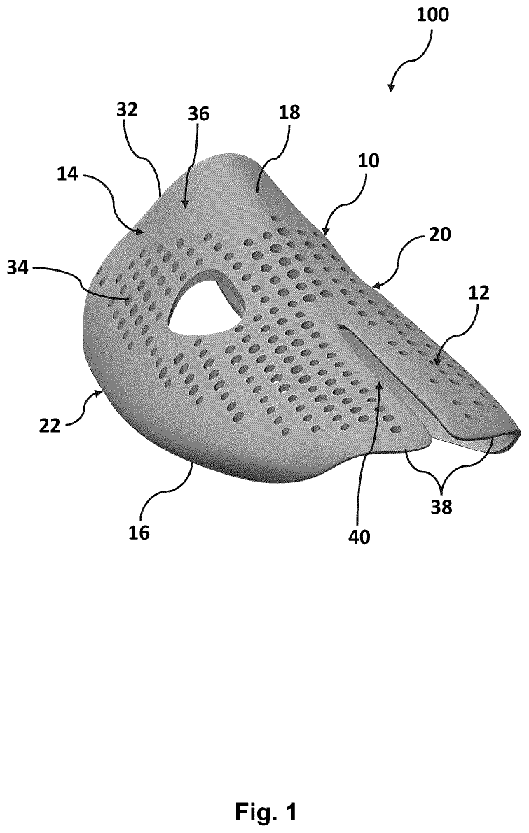

FIG. 1 depicts a perspective front view of a support shoe last according to an embodiment;

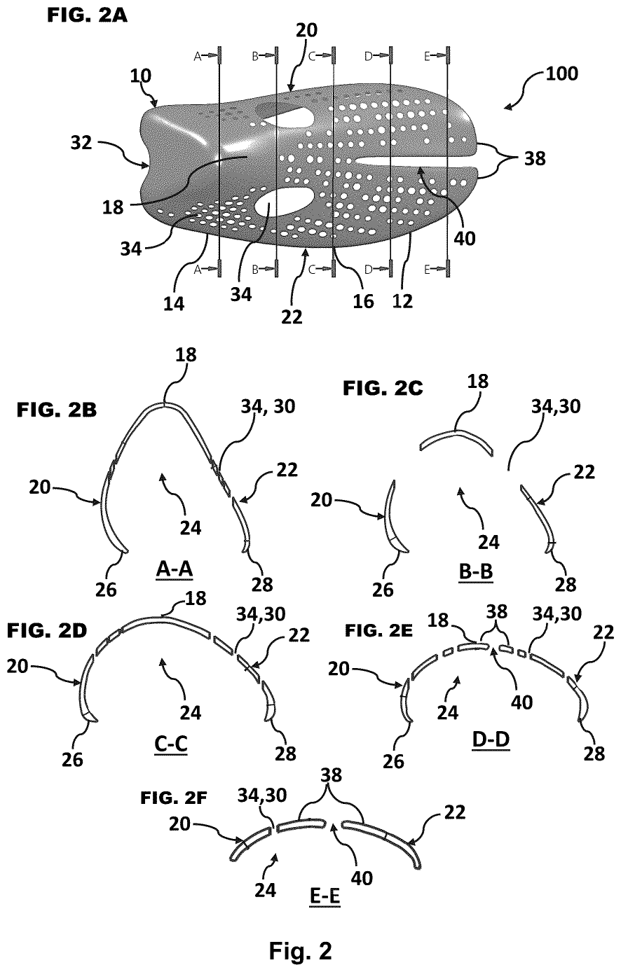

FIG. 2A depicts a top view of a support shoe according to an embodiment;

FIG. 2B depicts a cross section of the support shoe last of FIG. 2A at line A-A;

FIG. 2C depicts a cross section of the support shoe last of FIG. 2A at line B-B;

FIG. 2D depicts a cross section of the support shoe last of FIG. 2A at line C-C;

FIG. 2E depicts a cross section of the support shoe last of FIG. 2A at line D-D;

FIG. 2F depicts a cross section of the support shoe last of FIG. 2A at line E-E;

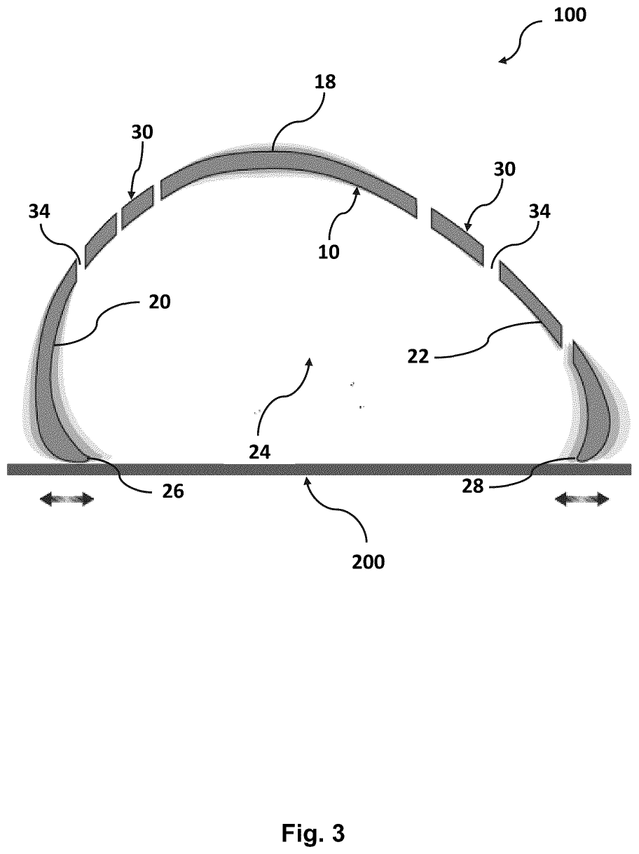

FIG. 3 depicts a cross section of a support shoe last with a landing pad engaging with the insole of a shoe according to an embodiment;

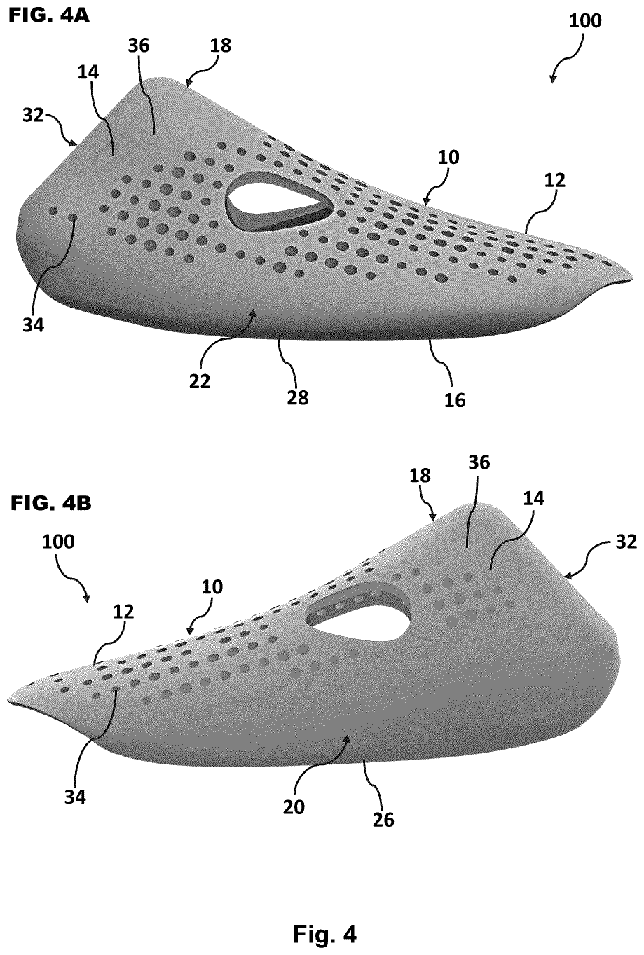

FIG. 4A depicts a side view of a support shoe last viewed from the outer side section according to an embodiment;

FIG. 4B depicts a side view of the support shoe last of FIG. 4A viewed from the arch side section;

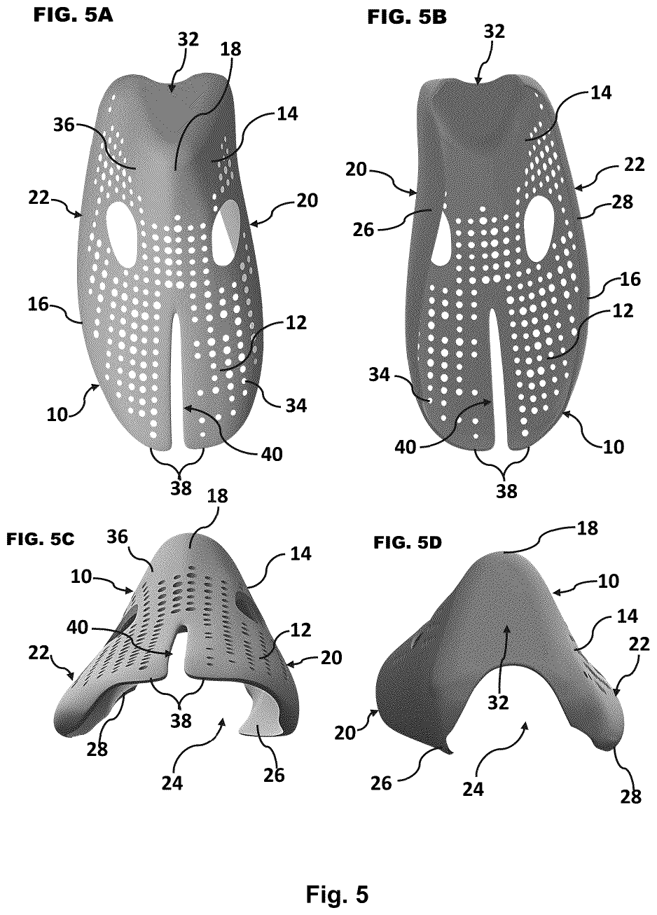

FIG. 5A depicts a top view of a support shoe last according to an embodiment;

FIG. 5B depicts a bottom view of the support shoe last of FIG. 5A;

FIG. 5C depicts a front view of the support shoe last of FIGS. 5A and 5B;

FIG. 5D depicts a rear view of the support shoe last of FIGS. 5A-5C;

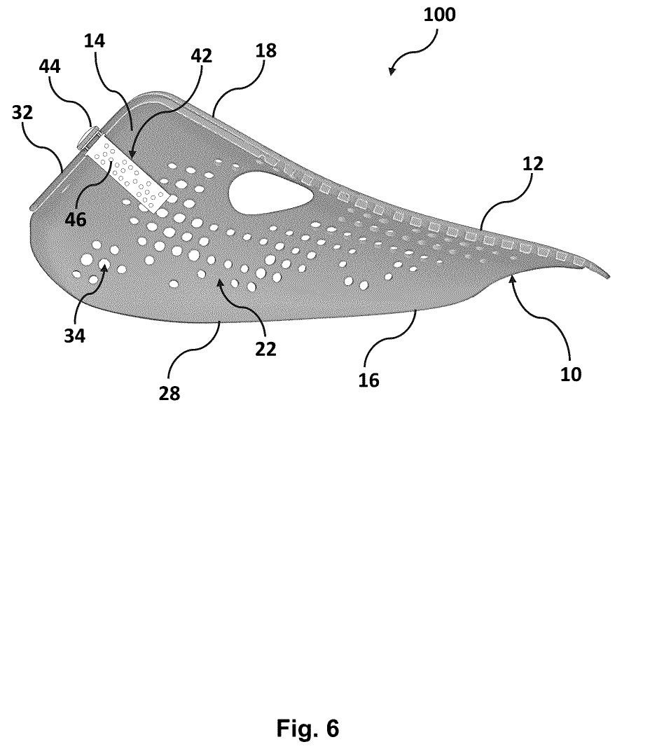

FIG. 6 depicts a side view of a support shoe last comprising a fragrance holder configured for holding a fragrance plug-in according to an embodiment;

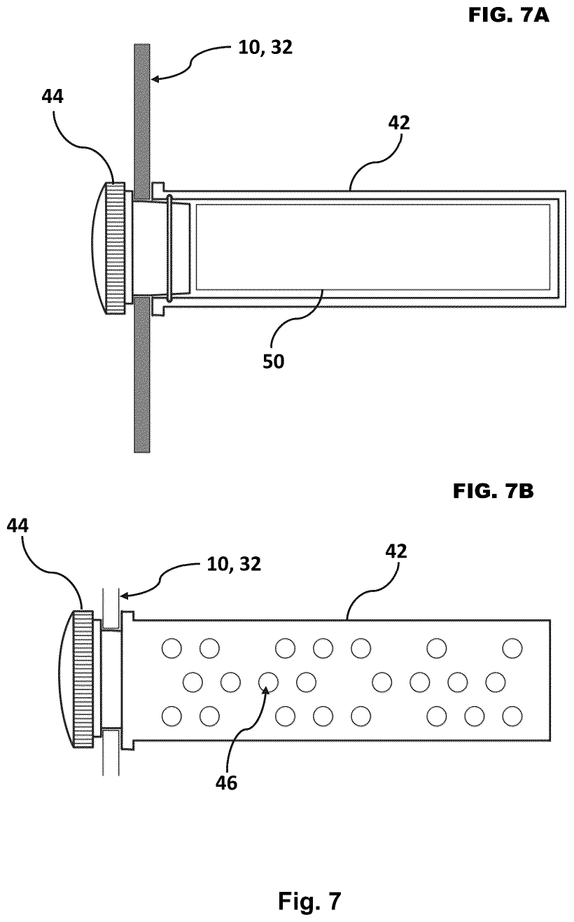

FIG. 7A depicts a view of a tube for holding a fragrance plug-in, illustrating a transparent view of the inside of the tube according to an embodiment;

FIG. 7B depicts a view of the tube for holding a fragrance plug-in, illustrating the outer surface of the tube according to an embodiment;

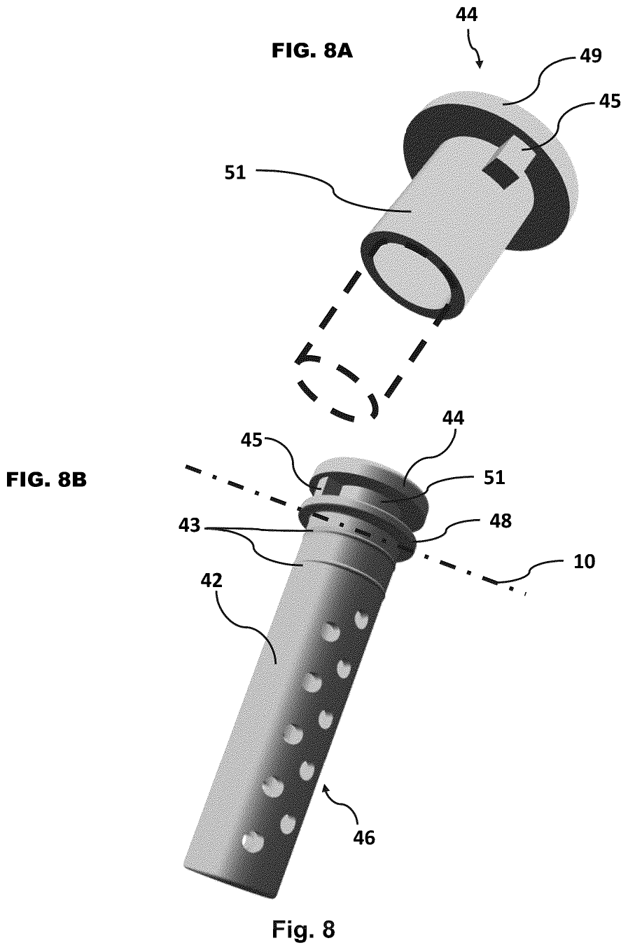

FIG. 8A depicts a cap and a fragrance stick of a fragrance holder according to an embodiment;

FIG. 8B depicts a tube and a cap of a fragrance holder according to an embodiment;

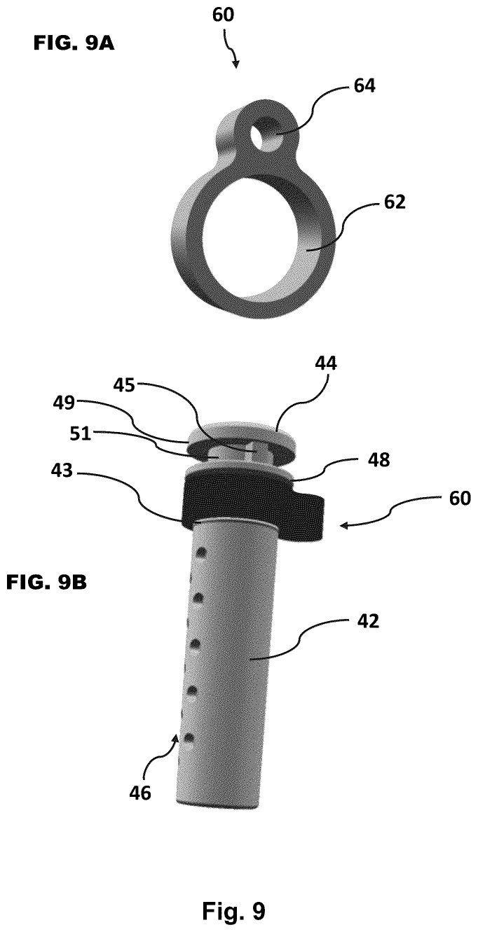

FIG. 9A depicts a cord connector according to an embodiment; and

FIG. 9B depicts the cord connector of FIG. 9A together with the fragrance holder of FIG. 8B.

LIST OF REFERENCE NUMBERS

| Item | No |
|---|---|
| Support shoe last | 100 |
| Shell body | 10 |
| Forepart | 12 |
| Waist part | 14 |
| Feather edge | 16 |
| Top section | 18 |
| Arch side section | 20 |
| Outer side section | 22 |
| Cavity | 24 |
| Arch landing pad | 26 |
| Second landing pad | 28 |
| Mechanical weakness | 30 |
| Rear side section | 32 |
| Perforations | 34 |
| Cone part | 36 |
| Split toe | 38 |
| Slit | 40 |
| Tube | 42 |
| Friction member | 43 |
| Cap | 44 |
| Stop member | 45 |
| Tube perforations | 46 |
| Tube flange | 48 |
| Fragrance plug-in | 50 |
| Channel | 51 |
| Fragrance stick | 52 |
| Cord connector | 60 |
| Sirst connector aperture | 62 |
| Second connector aperture | 64 |
| Insole | 200 |

### DETAILED DESCRIPTION

FIG. 1 illustrates a perspective front view of a support shoe last 100, where the support shoe last 100 comprises a shell body 10 that defines a forepart 12, a waist part 14 comprising a cone part 36, and a feather edge 16 between the forepart 12 and the waist part 14.

The shell body 10 may be constructed to simulate the anatomy of a front foot. The shell body 10 enables the support shoe last 100 to simultaneously support the shoe while the support shoe last 100 is deformed by the shoe such that the support shoe last 100 does not permute the shoe in an unnatural way.

The shell body further comprises a top section 18 extending from the waist part 14 to the forepart 12 of the shell body 10. An arch side section 20 and an outer side section 22 extends downwards from the top section 18, thereby defining a cavity 24 of the shell body 10. Thus, the arch side section 20 and the outer side section 22 extend downwards from the top 18 section towards the insole 200 of the shoe. Thereby, the shell body 10 of the support shoe last 100 is adapted to a curvature of the upper part of the front of the shoe.

The cavity 24 of the shell body 10 formed by the arch side section 20 and the outer side section 22 is hollow which enables the shell body 10 to adapt to the shape of the shoe i.e. the shell body 10 makes the support shoe last flexible, and thus adaptive to the original shape of the shoe.

An arch landing pad 26 extends from the arch side section 20 towards the outer side section 22 along part of the waist part 14 of the shell body 10, and a second landing pad 28 extends from the outer side section 22 towards the arch side section 20 along part of the waist part 14 of the shell body 14, wherein both the arch landing pad 26 and the second landing pad 26 substantially lie in the same horizontal plane and both landing pads 26,28 have an insole 200 facing side which are in contact with the insole 200 of the shoe. (See FIGS. 3 and 5A-5D).

The arch landing pad 26 and the second landing pad 28 may be asymmetrical in shape and in length. Thereby, the support shoe last 100 can simulate the anatomic shape of a foot which is asymmetrical, and thus simulate the shape of a shoe which is shaped after a foot, and therefore also asymmetric and anatomic.

The landing pads 26,28 may extend along part of the waist part 14 to the feather edge 16 and optionally along part of the forepart 12. The feather edge 16 is the transition between the waist part 14 and the forepart 12. In some embodiments only the second landing pad 28 will extend along part of the forepart 12, thereby forming an asymmetry between the two landing pads 26,28.

The forepart 12 may comprise a split toe 38 that is formed by a slit 40, wherein each side of the slit 40 are curved downwardly. Thereby, each side of the slit 40 will automatically move downwards and together and adjust to the shape of the toe of the shoe.

In some embodiments, the forepart 12 may comprise a toe spring.

The arch side section 20 and the outer side section each 22 comprise a mechanical weakness 30 extending longitudinal along at least part of the shell body 10, where the mechanical weakness 30 may be achieved by decreasing the material thickness of the shell body 10 on each side of the top section 18 at the joint between the top section 18 and each of the two side sections 20,22. Thereby enabling the arch section 20 and the outer side section 22 to bend and adjust to the shape of a shoe, and thus increasing the flexibility of the support shoe last 100 as various shoes will have different widths (see FIG. 3).

The mechanical weakness 30 may be formed by having less material thickness compared to the average material thickness of the side sections 20,22 and/or formed by micro-perforations or macro-perforations 34 along the side sections 20,22.

Forming the mechanical weakness 30 by decreasing the material thickness of the of the side sections 20,22 and/or making perforations 34 along the side sections 20,22 increases the flexibility of the bend of the two side sections 20,22, and less material is needed to produce the support shoe last 100.

The shell body 10 may further comprise at the waist part 14 and distal to the forepart 12 a rear side section 32 extends downwardly from the top section 18 and interconnects the arch side section 20 and the outer side section 22 (see FIG. 5D). This provides a stiffness to the shell body 10 and increases the stability wherein collapse of the support shoe last 100 under pressure is prevented.

The support shoe last 100 may be a one-piece construction without any hollow or delicate parts. It may further comprise of a thermoplastic material such as thermoplastic polyurethane or recycled thermoplastic polyurethane or plant-based thermoplastic polyurethane or polyethylene.

Thermoplastic polyurethane or recycled thermoplastic polyurethane or plant-based thermoplastic polyurethane may be used for making the support shoe last 100, as it is very flexible materials and can be made in different degrees of hardness. It is therefore possible to make varying thicknesses of the shell body 10, thereby creating mechanical weakness 30 to increase the flexibility of the support shoe last 100 without risking breakage.

In some embodiments the cone part 36 of the shell body 10 may have a higher surface roughness than the rest of the shell body 10, such that the support shoe last 100 is secured within the shoe.

The cone part 36 may be made of a softer thermoplastic polyurethane, and the rest of the shell body 10 may be made of a harder but still flexible thermoplastic polyurethane. The softer thermoplastic polyurethane may have a higher surface roughness than the harder thermoplastic polyurethane. Thus, the cone part 36 may provide friction between the support shoe last 100 and the inner lining of the shoe. Thereby, the support shoe last 100 holds itself in place within the shoe.

The harder thermoplastic polyurethane may have a smooth surface that slides smoothly along the inner lining of the shoe, which ensures an easy insertion and removal of the support shoe last 100.

The support shoe last 100 can be used for various shoe sizes as the dimensioning of the shoe support last 100 just needs to be adapted to the different shoe sizes.

FIGS. 2A-2F illustrate a top view of a support shoe last 100 and cross sections at five different positions, respectively. FIG. 2A shows a top view of a support shoe last 100 corresponding to the support shoe last 100 illustrated in FIG. 1.

FIG. 2B illustrates the cross-section A-A at a position on the waist part 14 of the support shoe last 100. The arch side section 20 and the outer side section 22 extends downwards from the top section 18, thereby defining a cavity 24 of the shell body 10. Thus, the arch side section 20 and the outer side section 22 extend downwards from the top 18 section towards the insole 200 of the shoe. The arch landing pad 26 in connection with the arch side section 20 may have a smaller radius of curvature towards the second landing pad 28, than the outer side section 22 and the second landing pad 28.

The outer side section 22 may be substantially straight deflecting in an outwards angle from the arch side section 20 with an inwards curvature in the connection with the second landing pad 28 towards the arch landing pad 26. The arch side section 20 may be substantially curved from the connection with the top section 18 to the arch landing pad 26.

Furthermore, the material of the support shoe last 100 may be thinner at the perforations 34, thereby providing the support shoe last 100 with a mechanical weakness 30.

FIG. 2C illustrates the cross-section B-B at a position on the waist part 14 of the support shoe last 100 further towards the forepart 12 than the cross-section A-A. The curvature of the top section 18 may be decreased compared to the top section 18 in FIG. 2B. Thus, the cavity 24 may become wider.

The material thickness of the support shoe last 100 may have increased at the arch landing pad 26. Furthermore, the large perforations 34 may further increase the mechanical weakness 30.

FIG. 2D illustrates the cross-section C-C at the feather edge 16 of the support shoe last 100. The curvature of top section 18 is further decreased compared to the top section 18 in FIGS. 2B and 2C. The support shoe last 100 may be widest at the feather edge 16.

The curvature of the outer side section 22 may be increased compared to the curvature of the outer side section 22 in FIGS. 2B and 2C. However, the curvature of the arch side section 20 may still be greater than the curvature of the outer side section 22.

Furthermore, the material thickness of the support shoe last 100 may have increased at the second landing pad 28, and the perforations 34 may provide mechanical weakness 30 in the support shoe last 100.

FIG. 2E illustrates the cross-section D-D at a position on the forepart 12 of the support shoe last 100. The curvature of the outer side section 22 is further increased compared to the curvature of the outer side section 22 in FIG. 2D.

The curvature of the arch landing pad 26 may have decreased along with the material thickness of the arch landing pad 26. Where the curvature and material thickness of the second landing pad 28 may be substantially the same compared to FIG. 2D. Thus, the second landing pad 28 may be longer than the arch landing pad 26 and extend longer along the forepart 12.

The slit 40 may create a slip toe 38 of the support shoe last 100. Thereby each side of the slit 40 may automatically move together and adjust to the shape of the toe of the shoe.

FIG. 2F illustrates the cross-section E-E at a position on the forepart 12 of the support shoe last 100 further towards the tip of the support shoe last 100 than the cross-section D-D. The curvature of the two side sections 20,22 may have decreased, and the two landing pads 26,28 may no longer be present.

The slit 40 may create a slip toe 38 of the support shoe last 100.

FIG. 3 illustrates a cross section of a support shoe last 100 with a landing pad 26,28 engaging with the insole 200 of a shoe.

The side sections 20, 22 may extend downwardly from the top section 18 towards the insole 200 of the shoe. Thereby, the shell body 10 of the support shoe last 100 follows the curvature of the upper part of the front of a shoe.

The cavity 24 of the shell body 10 formed by the arch side section 20 and the outer side section 22 is hollow which enables the shell body 10 to adapt to the shape of the shoe i.e. the shell body 10 makes the support shoe last 100 flexible, and thus adaptive to the original shape of the shoe.

The arch landing pad 26 and the second landing pad 28 extend from the arch side section 20 and second side section 22 respectively towards the insole 200 of the shoe, where the landing pads 26, 28 engage with the insole 200.

The landing pads 26,28 may comprise curved outer surfaces, which curved outer surfaces are curved inwardly towards the cavity 24. Thereby, the landing pads 26,28, the arch side section 20, and outer side section 22 will automatically deform inwardly towards the cavity 24 of the shell body 10. Thus, when the support shoe last 100 is inserted in the shoe, the landing pads 26,28 automatically squeezes together, due to the inward curvature, to adapt to the volume and fit of the shoe.

In some embodiments, the landing pads 26,28 may be curved upwardly. The upwardly curved landing pads 26,28 may slide together when the support shoe last 100 is inserted into a shoe, thereby adjusting the support shoe last 100 to the width and height of the shoe. The upward curvature further ensures that the two landing pads 26,28 do not collide with each other when pushed together inside the shoe.

In an embodiment, the landing pads 26, 28 may be rounded such that they may slide together when the support shoe last 100 is inserted into a shoe, thereby adjusting the support shoe last 100 to the width and height of the shoe. Furthermore, the rounded landing pads 26, 28 may enable a smooth insertion of the support shoe last 100 into the shoe, where scratching of the insole 200 of the shoe and the support shoe last 100 getting stuck is avoided.

Both side sections 20, 22 comprise a mechanical weakness 30 extending longitudinal along at least part of the shell body 10, where the mechanical weakness 30 may be achieved by decreasing the material thickness of the shell body 10 on each side of the top section 18 at the joint between the top section 18 and each of the two side sections 20, 22. The mechanical weakness 30 may also be formed by micro-perforations or macro-perforations 34 distributed along the side sections 20,22). The mechanical weakness may increase the flexibility of the support shoe last 100.

The mechanical weakness 30 enables the arch side section 20 and the outer side section 22 to bend inwards towards each other, thereby moving the arch landing pad 26 and the second landing pad 28 towards each other. This is illustrated with the shaded areas in the figure. When the side sections 20,22 bend, the curvature of the top section 18 may increase or decrease resulting in the top section 18 being displaced away from or towards the insole 200 of the shoe respectively. Thus, when the side sections 20,22 bend inwards towards each other, the curvature of the top section 18 increases and the top section 18 is displaced away from the insole 200 of the shoe. When the side sections 20, 22 move away from each other, the curvature of the top section 18 decreases and the top section 18 is displaced towards the insole 200 of the shoe.

The mechanical weakness 30 further enables the support shoe last 100 to adjust to the shape of a shoe. Thus, when inserting the support shoe last 100 into the shoe, the arch landing pad 26 and the second landing pad 28 will automatically slide together and adjust to the width, height, volume, and fit of the shoe. Thereby adapting to and imitating the shape of the shoe.

In some embodiments the mechanical weakness 30 of each side section 20, 22 may extend longitudinal along the entire shell body 10. However, this will also decrease the biasing force of the support shoe last 100, and this will increase the risk of the hollow shell body 10 collapsing, thus these embodiments may be less desired.

In some embodiments the mechanical weakness 30 may extend longitudinal along a part of the shell body 10 between the feather edge 16 to the waist part 14. Thereby increasing the stability of the support shoe last 100 and preventing collapse of the support shoe last 100 under pressure, and still providing the necessary flexibility to the support shoe last 100.

FIGS. 4A and 4B illustrate two side views of a support shoe last 100, where FIG. 4A is a side view of the outer side section 22 and FIG. 4B is a side view of the arch side section 20. The support shoe last 100 illustrated in FIG. 4 corresponds to the support shoe last 100 illustrated in FIG. 1.

FIGS. 5A-5D illustrate four different views of a support shoe last 100.

FIG. 5A is a top view of a support shoe last 100 corresponding to the support shoe last 100 illustrated in FIG. 1.

FIG. 5B is a bottom view of a support shoe last 100 corresponding to the support shoe last 100 illustrated in FIG. 1.

FIG. 5C is a front view of a support shoe last 100 corresponding to the support shoe last 100 illustrated in FIG. 1. FIG. 5C further illustrates the landing pads 26, 28, which may comprise curved outer surfaces, which curved outer surfaces are curved inwardly towards the cavity 24. Thereby, the landing pads 26,28, the arch side section 20 and outer side section 22 will automatically deform inwardly towards the cavity 24 of the shell body 10 (see also FIG. 5D).

FIG. 5D a rear view of a support shoe last 100 corresponding to the support shoe last 100 illustrated in FIG. 1. FIG. 5D further illustrates the rear side section 32, which may further be constructed so that a gap is obtained between the rear side section 32 and the insole 200 of the shoe, thereby enabling the rear side section 32 to act as a handle, that two fingers may grab around for easy removal of the support shoe last 100 from the shoe.

In some embodiments the top of the rear side section 32 may be constructed with a tip (not shown) peaking at a lower point than the highest point of the top section 18. This tip may not be configured to interact with the shoe and thus not serve a support purpose in the shoe. Instead, it eases the removal of the support shoe last 100 from the shoe as a finger may grab around the tip, thereby providing a better grip when pulling the support shoe last 100 out of the shoe.

FIG. 6 illustrates a side view of a support shoe last 100 comprising a tube 42 configured for holding a fragrance plug-in 50. The support shoe last 100 corresponds to the support shoe last 100 illustrated in FIGS. 1-5D.

The shell body 10 may further comprise a fragrance holder configured to hold a fragrance plug-in 50.

The fragrance holder may be positioned at the rear side section 32 and optionally the fragrance holder may comprise a tube 42 extending through the rear side section 32, thereby making the fragrance holder accessible even when the support shoe last 100 is supporting a shoe.

The tube 42 may be configured for holding a fragrance plug-in 50, and the opening of the tube 42 may be positioned at the rear side section 32.

The tube 42 may comprise a cap 44 for closing of the opening of the tube 42 and keeping the fragrance plug-in 50 secured inside the tube. The tube 42 may further comprise tube perforations 46 enabling diffusion of fragrance from the fragrance plug-in 50 inside the tube 42 to the shoe.

In some embodiments the tube 42 may be cast into the shell body 10.

FIGS. 7A and 7B illustrate two different views of a tube 42 for holding a fragrance plug-in 50. FIG. 7A illustrates a transparent view, where the inside of the tube 42 is visualized. In the illustrated embodiment, the tube 42 is embedded in the shell body 10 of the support shoe last 100 with the tube opening at the surface of the shell body 10. The tube 42 may be placed in the shell body 10 such that the tube opening is at the rear side section 32.

The tube 42 may comprise a cap 44 for closing the tube 42 and keeping the fragrance plug-in 50 secured inside the tube 42.

In some embodiments the cap 44 may be permanently connected to the tube 42 through at strip, such that misplacement of the cap 44 is avoided. In embodiments the cap 44 may be a loose cap 44.

FIG. 7B illustrates the outer surface of the tube 42 described above for FIG. 7A. The tube 42 may further comprise tube perforations 46 enabling diffusion of fragrance from the fragrance plug-in 50 inside the tube 42 to the shoe.

FIGS. 8A and 8B illustrate a fragrance holder. FIG. 8A shows a cap 44 and a fragrance stick is illustrated by the dashed cylinder and FIG. 8B shows the tube 42 and cap 44.

The fragrance holder has a tube 42 with tube perforations 46 and a cap 44 comprising a cap cylinder 51 extending from a cap flange 49, wherein the cap flange (49) having a greater width than the cap cylinder 51.

The cap cylinder 51 has a channel adapted for removable holding a fragrance stick. The cap cylinder 51 is configured to frictionally engage the tube 42 as shown in FIG. 8B.

The cap 44 has a stop member 45 such that there is distance between the cap flange 49, and the tube 42 when the cap 44 is inserted partly into the tube 42 as shown in FIG. 8B.

The tube has a tube flange 48 for engaging a shell body 10 or a cord connector 60 as shown in FIGS. 9A and 9B.

The tube has two external friction members 43 for frictionally engaging the aperture of a shell body 10 or a cord connector 60. In the shown embodiment, the two external friction members 43 are ring protrusions positioned with a mutual distance substantially equal to the thickness of a cord connector as shown in FIG. 9B.

FIGS. 9A and 9B illustrate a fragrance holder and a cord connector 60. The shown fragrance holder in FIG. 9B is the same fragrance holder shown in FIG. 8B.

The cord connector 60 has a first connector aperture 62 adapted for frictionally engaging the tube 42 and a second connector aperture 64 adapted for engaging a cord.

Although the present invention has been disclosed in the form of embodiments and variations thereon, it will be understood that numerous additional modifications and variations could be made thereto without departing from the scope of the invention.

For the sake of clarity, it is to be understood that the use of “a” or “an” throughout this application does not exclude a plurality, and “comprising” does not exclude other steps or elements. The mention of a “unit” or a “module” does not preclude the use of more than one unit or module.

1. A support shoe last for supporting a shoe on display or during transportation, the support shoe last comprising:
a shell body defining a forepart, a waist part comprising a cone part, and a feather edge between the forepart and the waist part, the shell body including:
a top section extending from the waist part to forepart;
an arch side section and an outer side section extending downwards from the top section, thereby defining a cavity of the shell body; and
an arch landing pad configured to engage an insole of a shoe,
where the arch landing pad extends from part of the arch side section and widens towards the insole,
a second landing pad configured to engage an insole of a shoe, the second landing pad extends from part of the outer side section and widens towards the insole,
wherein the arch side section and/or the outer side section comprise a mechanical weakness extending longitudinal along at least part of the shell body, thereby enabling the arch section and the outer side section to bend and adjust to the shape of a shoe.

2. The support shoe last according to claim 1, wherein at least one of the arch landing pad and the second landing pad extends along part of the waist part to the feather edge and optionally along part of the forepart.

3. The support shoe last according to claim 1, wherein at least one of arch landing pad and the second landing pads comprise curved outer surfaces, which curved outer surfaces are curved inwardly towards the cavity.

4. The support shoe last according to claim 2, wherein the curvature of the outer surface of the second landing path is greater than the curvature of the outer surface of the arch landing path.

5. (canceled)

6. The support shoe last according to claim 1, wherein the mechanical weakness is formed by having less material thickness compared to an average material thickness of the side sections and/or formed by micro-perforations or macro-perforations along the side sections.

7. (canceled)

8. (canceled)

9. (canceled)

10. The support shoe last according to claim 1, wherein the shell body further comprises a fragrance holder configured to hold a fragrance plug-in.

11. The support shoe last according to claim 10, wherein the fragrance holder is positioned at the rear side section and optionally the fragrance holder comprises a perforated tube extending through the rear side section, thereby making the fragrance holder accessible even when the support shoe last is supporting a shoe.

12. The support shoe last according to claim 10, wherein the fragrance holder extends through an aperture of the shell body.

13. The support shoe last according to claim 10, wherein the fragrance holder comprises
a tube with tube perforations, and
a cap comprising a cap cylinder extending from a cap flange, wherein the cap flange having a greater width than the cap cylinder,
wherein the cap cylinder comprises a channel configured for removably holding a fragrance stick, wherein the cap cylinder is configured to frictionally engage the tube.

14. The support shoe last according to claim 13, wherein the cap and/or the tube comprises a stop member such that there is distance between the cap flange and the tube when the cap is inserted partly into the tube.

15. The support shoe last according to claim 13, wherein the tube comprises a tube flange for engaging the shell body.

16. The support shoe last according to claim 13, wherein the tube comprises one or more external friction members for frictionally engaging the aperture of the shell body.

17. A fragrance holder for removeably holding a fragrance stick, the fragrance holder comprising:
a tube with tube perforations, and
a cap comprising a cap cylinder extending from a cap flange, wherein the cap flange having a greater width than the cap cylinder,
wherein the cap cylinder comprising a channel configured for removably holding the fragrance stick, wherein the cap cylinder is configured to frictionally engage the tube.

18. The fragrance holder according to claim 17, wherein the cap and/or the tube comprises a stop member such that there is distance between the cap flange and the tube when the cap is inserted partly into the tube.

19. (canceled)

20. The fragrance holder according to claim 17, wherein the tube comprises one or more external friction members for frictionally engaging the aperture of a shell body.

21. The fragrance holder according to claim 17, wherein the fragrance holder comprises a cord connector comprising a first connector aperture configured for frictionally engaging the tube and a second connector aperture configured for engaging a cord.
